(19) United States
(12) Patent Application Publication
(10) Pub. No.: US 2012/0021145 A1
(43) Pub. Date: Jan. 26, 2012

(54) Biodegradable Sorbent Devices For Use In Liquid Spills

(76) Inventors: Craig S. Cargill, Kemah, TX (US); Jerry L. McGinty, Austin, TX (US); Michael A. Magnoli, Mobile, AL (US)

(21) Appl. No.: 13/219,977

(22) Filed: Aug. 29, 2011

Related U.S. Application Data
(60) Provisional application No. 61/379,534, filed on Sep. 2, 2010.

(51) Int. Cl. B01J 20/28; B01J 20/22; C08F 110/06; D03D 25/00; D04D 7/06; B32B 5/02; B32B 1/02; B01J 20/26; A01H 5/00

(52) U.S. Cl. 428/4; 502/400; 502/402; 502/401; 526/351; 800/298; 428/221; 442/1; 428/34.1; 442/181

(57) ABSTRACT

The present invention relates to the development and manufacture of sorbent products and devices suitable for use in hydrocarbon, universal and hazmat applications that are composed of structural components and/or sorbent materials that are fully biodegradable and landfill friendly. The structural components and/or sorbents may be derived from biodegradable polypropylene or biodegradable natural materials and combined in different sizes, shapes and configurations.

### REFERENCE TO RELATED APPLICATIONS

This application claims priority to United States Provisional Patent Application No. 61/379,534, filed Sep. 2, 2010, the contents of which are herein incorporated by reference in its entirety.

### TECHNICAL FIELD

The present invention relates to the development and manufacture of sorbent devices that are composed of structural components and/or sorbent materials that are fully biodegradable and landfill friendly. Such devices are suitable for use in liquid spills involving hydrocarbons, as well as universal and hazmat applications involving aggressive or non-aggressive liquids.

### BACKGROUND OF THE INVENTION

Spill of such hydrocarbons such as crude oil, refined oil, kerosene, gasoline and machine oil, and more aggressive liquids such as acids, bases and chlorinated solvents, occur daily, creating minor to major environmental pollution. These occur both on land and in aquatic environments. Aquatic spills include releases of crude oil from tankers, offshore platforms, drilling rigs and wells as well as oily refuse or waste oil from a variety of commercial operations. On land, spills occur as a result of operations at refineries, during transportation of aggressive liquids and at a variety of manufacturing facilities and are a result of both natural and man-made disasters.

The containment, adsorption and removal of the hydrocarbons and/or aggressive liquids involved in such spills is critical to minimizing damage to fragile ecosystems and wildlife. For years, sorbent products configured as booms, bilge booms, socks, mats, rolls, pillows and pom-poms, etc., have proven to be cost effective in containing, adsorbing and removing spilled hydrocarbons. However, approximately 98% all sorbent products, particularly hydrocarbon sorbent products, have been constructed using non-biodegradable polypropylene. Such products use non-biodegradable polypropylene filaments as the sorbent, non-biodegradable polypropylene ropes for connection, non-biodegradable polypropylene casings to enclose the sorbent and non-biodegradable polypropylene scrims to protect the casings. In essence, the entire product, with exception of the metal connectors, is non-biodegradable plastic which accumulates in the environment and becomes a serious source of pollution for flora and fauna.

Each year the use of non-biodegradable plastics generates approximately 25 million tons of waste which persists for 200 years or more in the environment because it will not decompose in land-fills. Polypropylene accounts for approximately 18% of this total. During a single oil spill, the BP PLC Deepwater Horizon spill in the Gulf of Mexico in 2010, over ten million pounds of non-biodegradable polypropylene booms were discarded into landfills in Louisiana, Mississippi, Alabama and Florida.

In an effort to reduce this non-biodegradable polypropylene waste stream, there have been several attempts over the years to use various combinations of natural products as sorbents, including kenaf, sugar cane stems, corn cob, hay, cellulose, cotton and straw. However, these products still rely on structural elements (e.g., ropes and casings and scrims) made from non-biodegradable polypropylene, thus greatly reducing the desired environmental benefits. All of these natural materials have floatation problems because they soak up large amounts of water which reduces their ability to adsorb hydrocarbons and makes them extremely heavy to retrieve after use. In addition, such natural materials leach sugars and other water-soluble materials into the aquatic habitats, creating a different set of environmental problems. One of the most severe problems with these natural sorbent materials is that they rapidly decay after deployment in aquatic habitats and create major odor and visual issues.

Thus, there is a need for improved sorbent products that effectively contain and/or adsorb hydrocarbons, aggressive liquids, and other liquids, without contributing to environmental pollution or imbalance.

### SUMMARY

The present invention provides sorbent products and devices for containing and/or adsorbing hydrocarbons, such as crude oil, refined oil, kerosene, gasoline, machine oil, or other hydrocarbons, aggressive liquids such as alcohols, acids, bases, ketones, solvents (e.g., chlorinated solvents), and detergents, as well as non-aggressive liquids such as milk, blood, water, juice, etc. The sorbent products/devices of the invention are composed of structural components (identified herein as ropes, casings and scrims) and/or sorbents, that are fully biodegradable, and thus are more environmentally and landfill friendly than sorbent products that only utilize traditional non-biodegradable materials as sorbents, such as non-biodegradable polypropylene, or products that utilize natural materials as sorbents, but include structural components (e.g., ropes, casings and/or scrims) that are non-biodegradable.

In one aspect, the invention provides devices for containing and adsorbing hydrocarbons, and other aggressive or non-aggressive liquids, in both aquatic and terrestrial environments, including indoor and outdoor environments, wherein one or more structural components (e.g., ropes, casings and/or scrims) are biodegradable. Examples of devices for containing and/or adsorbing hydrocarbons or other aggressive or non-aggressive liquids involved in aquatic or land-based spills include, but are not limited to, booms, bilge booms, socks, dikes, mats, rolls, pads, mops, rugs, pillows, pom-poms, skimmers, sweeps and wipes. It should be noted that not all such devices require ropes, casings and scrims, in combination. Each of these structural components may be included in a particular sorbent device alone, or in combination with one or more of such structural components. However, any of such structural components that may be included in a hydrocarbon sorbent device of the invention, shall be biodegradable.

In certain embodiments, the structural components of the sorbent products/devices according to the invention are made from biodegradable polypropylene. In other embodiments, the structural components are made from natural, biodegradable material such as burlap, jute, hemp, sisal, or any combination thereof.

The sorbent products/devices including one or more biodegradable structural components according to the invention can further include a biodegradable sorbent. In certain embodiments, the biodegradable sorbent is composed of biodegradable polypropylene. In other embodiments, the biodegradable sorbent is a combination of raw short-fiber cotton and perlite, or a combination of biodegradable polypropylene, raw short-fiber cotton and/or perlite.

In some embodiments, the sorbent products/devices including one or more biodegradable structural components in accordance with the invention can further include one or more hydrocarbon degrading bacteria, or any microbial compound approved by the Environmental Protection Agency for use in hydrocarbon degradation.

In another aspect, the invention provides biodegradable sorbents for use in sorbent products/devices. In certain embodiments, the sorbents are composed of biodegradable polypropylene. In other certain embodiments, the sorbents are composed of a combination or raw short-fiber cotton and perlite, or a combination of biodegradable polypropylene, raw short-fiber cotton and/or perlite.

In yet another aspect, the invention provides sorbent products/devices for containing and adsorbing hydrocarbons, aggressive liquids, or non-aggressive liquids, from terrestrial (indoor and outdoor) and aquatic environments that include one or more structural components and a sorbent, each of which biodegradable. Such products/devices include, but are not limited to, booms, bilge booms, socks, dikes, mats, rolls, pads, mops, rugs, pillows, pom-poms, skimmers, sweeps and wipes. The biodegradable structural components can be made from biodegradable polypropylene or other natural, biodegradable material such as burlap, jute, hemp, or sisal, or any combination thereof. The biodegradable sorbents can be composed of biodegradable polypropylene, a combination or raw short-fiber cotton and perlite, or a combination of biodegradable polypropylene, raw short-fiber cotton and/or perlite.

In some aspects, the sorbent products/devices according to the invention include one or more connectors, or connecting components that are composed of metal. For example, a boom according to the invention may include one or more biodegradable structural components (e.g., ropes, casings and/or scrims) and/or a biodegradable sorbent, and metal connectors to facilitate the linking/connection of multiple booms together. The metal connectors are removable and thus can be removed prior to disposing the boom in a land-fill, thereby providing a sorbent device that is fully biodegradable. In other certain embodiments, the sorbent products/devices according to the invention include connecting components that are biodegradable. For example, the connecting component can be composed of a biodegradable plastic, or a suitable natural material.

Various aspects, features, objects, advantages, and details of the invention herein disclosed will become apparent through reference to the following description and the claims.

### BRIEF DESCRIPTION OF THE DRAWINGS

The present invention will be more fully understood from the following detailed description of illustrative embodiments taken in conjunction with the accompanying drawings. The illustrations in the drawings are not necessarily drawn to scale, the emphasis instead being placed generally on illustrating the principles of the invention and the disclosed embodiments.

FIG. 1 is an end view cross section of a biodegradable sorbent boom according to an exemplary embodiment of the present invention.

FIG. 2 is a lengthwise cross section of a biodegradable sorbent boom according to an exemplary embodiment of the present invention.

FIG. 3 is an end view cross section of a biodegradable sorbent sock according to an exemplary embodiment of the present invention.

FIG. 4 is a lengthwise cross section of a biodegradable sorbent sock according to an exemplary embodiment of the present invention.

### DETAILED DESCRIPTION

In order to facilitate an understanding of the present invention, a number of terms are defined below for use in this Description and the appended Claims.

As used herein, the term “adsorption” refers to the accumulation of gases, liquids, or solutes on the surface of a solid or liquid.

As used herein, the term “aggressive liquid(s)” refers to liquids such as alcohols, acids, bases, ketones, solvents (e.g., chlorinated solvents), detergents, or other liquid chemicals that may be environmentally hazardous or harmful if ingested or contacted with an external or interior surface or cavity of a person or other living being.

As used herein, the term “non-aggressive liquid(s)” refers to liquids such as water, blood, milk, juice, etc., or any liquid that is environmentally friendly or inert, or that does not pose a threat or danger to the well being of a person or other living being.

As used herein, the term “universal application” refers to applications or situations involving “non-aggressive liquid” spills.

As used herein, the term “hazmat application” refers to applications or situations involving “aggressive liquid” spills.

As used herein, the term “polypropylene” refers to either 1) any of various thermoplastic resins that are polymers of propylene. They are used to make molded articles and fibers, or 2) a fabric of fibers made of any of these resins.

As used herein, the term “biodegradable polypropylene” refers to polypropylene that will degrade in natural aerobic or anaerobic environments from the action of naturally occurring microorganisms (e.g., bacteria, fungi, algae), as measured by ASTM D5209-91, ASTM 5511, ASTM 5511-02, or ASTM D5526-94 (2002) Standard Test Methods.

As used herein, the term “boom(s)” refers to flexible cylindrical products containing materials that tend to attract and hold oil through the process of adherence. These are typically deployed in aquatic areas (marshes, shorelines, rivers, lakes, etc) after an oil spill or in anticipation of such an environmental problem. They typically contain a rope that runs the length of the product and connectors so that each length of boom can attach to another in an over-lapping manner and eliminate issues associated with “by-pass.”

As used herein, the term “bilge boom” refers to booms that are deployed in the bilges of boats and ships to adsorb any gasoline, oil, transmission fluid or other hydrocarbons that may settle in such areas. Bilge booms are cylindrical and typically have a rope looped at one end or both to facilitate removal from the bilge.

As used herein, the term “sorbent” refers to an internal material in a sorbent product, such as a boom, a bilge boom, a sock, a mat, a roll, a pad, a mop, a rug, a pillow, or a pom-pom, that “adsorbs” the hydrocarbons.

As used herein, the term “pillow(s)” refers to pillow shaped sorbent products/devices, with or without ropes, normally deployed in enclosed aquatic environments such as tanks, wells, etc.

As used herein, the terms “pom-pom(s)”, “mop(s)”, “sweep(s)”, “wipe(s)” and “skimmer(s)” refer to sorbent products/devices composed of loose strands of sorbent materials and are typically utilized in both terrestrial (indoor and outdoor) and aquatic environments.

As used herein, the terms “sock(s)” and “dike(s) refer to flexible cylindrical products/devices containing sorbent materials that are typically deployed in such land based areas as factories, refineries, production facilities, and road construction zones to mitigate problems with hydrocarbon spills and leaks. Socks typically contain the same general configuration as booms, including a casing an inner sorbent, but without ropes, connectors (e.g., metal or biodegradable connectors) or scrims, which are typically included in booms.

As used herein, the terms “mat(s)”, “pad(s)”, “roll(s)”, and “rugs” refer to sorbent products/devices with low profiles and are used primarily to adsorb hydrocarbon drips or small spills. They may be deployed both on land (indoor and outdoor) an in aquatic situations.

As used herein, the term “sorbent” refers to a solid material that accumulates another material on its surface. Sorbents are typically included as an internal material within a sorbent product or device. In some cases, such as the production of pom-poms, mat, rolls and rugs, the product itself may be the sorbent without any additional “internal” materials.

As used herein, the phrase “structural component(s)”, in reference to sorbent products/devices, refers to one or more of the following: (1) a protective exterior scrim, usually in the form of a woven or molded netting, (2) a casing that encloses the material(s) used to adsorb the liquid, and (3) a rope running the length of the product to assist in linking sections of a sorbent product/device (e.g., a boom) together. As used herein, the term “structural component(s)” is distinct from connectors or connecting components that may be used to connect or link sorbent products/devices together.

As used herein the term “bole” refers to the fruiting structure of a cotton plant in which the cotton seeds and lint grows.

As used herein, the terms “cotton gin waste”, “cotton gin trash” or “cotton gin residue” (CGR) each refer to material composed of the bole material, burrs, leaves, stalks and other residue of the cotton plant, seeds, twigs and dirt left at the gin after the cotton has been processed. (It does not include whole seed.)

As used herein, the terms “gin motes” or “motes” refer to cotton fibers that are reclaimed from cotton ginning waste, and may be either fuzzy (immature seeds on which fiber development ceased at an early stage) or bearded (a piece of seed coat with long fibers attached).

As used herein the term “ginning” refers to the first processing of agricultural cotton in its natural form to separate the spinnable cotton fibers (lint) from the seeds and other material. Generally, only very short-fibers (linters) remain on the cotton seed after ginning.

As used herein, the term “lint” refers to the cotton fiber obtained by the ginning process once the cotton seed, leaves and casings have been removed.

As used herein, the term “mote ginning” refers to the process by which raw motes (leaves, trash, sticks, dirt, and immature cotton with some cotton seed) are run through a ginning process to extract the short-fiber cotton

As used herein, the term “reginned cotton” refers to cotton that has passed through the ginning process more than once. A bale of cotton is considered reginned when it is opened after the initial ginning and baling for the purpose of putting the lint through machinery to regin, clean, blend, or otherwise process the lint, and is then rebaled. Cotton which passes through one or more stages of lint cleaning as a regular part of the initial cleaning process is not reginned.

As used herein the term “seed cotton” refers to unginned, picked cotton in its natural state prior to any processing.

As used herein, the term “short-fiber cotton” refers to cotton produced by the separation of short-fibers or fuzz from cottonseed. This process can occur either in the processing (reginning) of cotton seed remaining from the first ginning process or from processing (reginning) cotton gin material known as cotton gin trash, cotton gin residue or cotton gin waste. This latter process is often referred to as “mote ginning.”

As used herein, the term “trash” refers to the leaf content of ginned cotton.

The present invention relates to the development and manufacture of sorbent products/devices. In particular, the present invention relates to sorbent products/devices, including but not limited to, booms, bilge booms, socks, dikes, mats, rolls, pads, mops, rugs, pillows, pom-poms, skimmers, sweeps, wipes, etc., useful for adsorbing and/or containing hydrocarbon, aggressive or non-aggressive liquid spills.

The sorbent products/devices of the invention are composed of one or more structural components (e.g., ropes, casings and/or scrims) and/or sorbent materials that are biodegradable and landfill friendly. The structural components may be derived from biodegradable polypropylene or natural materials such as burlap (e.g., wide weave burlap), jute, hemp, and/or sisal. The sorbent materials may be derived from biodegradable polypropylene or a combination of raw short-fiber cotton and perlite. All of these configurations are biodegradable and landfill friendly.

The present invention allows for multiple combinations of biodegradable structural components (ropes, casings and/or scrims) and biodegradable sorbents to allow a variety of products of various sizes, shapes, and configurations, to be manufactured for universal and hazmat sorbent applications, as described in further detail below.

Biodegradable Polypropylene

The present invention is based, in part, on the discovery that biodegradable polypropylene is suitable for use in sorbent products/devices, including but not limited to booms, bilge booms, socks, dikes, mats, rolls, pads, mops, rugs, pillows, pom-poms, sweeps, skimmers and wipes. Surprisingly, such products/devices including structural components and/or a sorbent composed of biodegradable polypropylene, in whole or in part, are substantially equal in weight, density, adsorbency (e.g., hydrocarbon adsorbency), and water uptake and flotation properties, as products made from traditional, non-biodegradable materials such as non-biodegradable polypropylene. Additionally, in contrast to products/devices that employ natural materials as sorbents, the sorbent products/devices of the invention do not rapidly decay upon deployment or create environmental problems by leaching sugars and other organic materials into the environment.

In recent years, substantial research has yielded an understanding of how the degradation of polypropylene can be directly or indirectly influenced through biological means. Biodegradation is a process which occurs due to the action of enzymes that are secreted by microbial organisms such as bacteria, fungi and/or algae, leading to chemical decomposition. Polypropylene's high molecular weight, polymer structure and hydrophobic nature affect its ability to be biodegraded. However, various techniques are now being employed to create biodegradable polypropylene. For example, it has been shown that the addition of additives, including but not limited to a variety of starch and non starch based products, to the resin of the polypropylene during the manufacturing process, will speed the disintegration of polypropylene plastics by oxy-bio degradation, oxidation and/or photodegradation. Thus, by blending such natural polymers as starch or cellulose with the synthetic polymers of the polypropylene, or by mixing the resin for producing polypropylene with pro-oxidants, a biodegradable form of polypropylene can be achieved. Additionally, biodegradable polypropylene can be produced by altering the surface of the polypropylene to induce the attachments of microorganisms. Each of these methods are designed to break the large polymer chains of polypropylene into structures small enough for microorganisms to use as a food source.

It should be noted that the present invention focuses on the use of biodegradable polypropylene in sorbent products and is not limited to one particular source, method, or chemical procedure for creating or producing biodegradable polypropylene.

Biodegradable polypropylene is currently being used in such products as disposable tableware, trash bags and other products. However, has not been employed for use in sorbent products/devices prior to the instant invention. The use of biodegradable polypropylene in the manufacture of structural components and/or sorbents for sorbent products and devices will greatly reduce the negative impact to landfills currently resulting from the disposal of used sorbent products made from non-biodegradable polypropylene.

Biodegradable and Landfill Friendly Structural Components

The present invention provides for the use of biodegradable structural components (e.g., ropes, casings, scrims, etc.) in sorbent products and devices that are suitable for use in hydrocarbon, universal and/or hazmat applications (e.g., booms, bilge booms, socks, dikes, mats, rolls, pads, mops, rugs, pillows, pom-poms, skimmers, sweeps and wipes). The biodegradable structural components can be made of biodegradable polypropylene, or other biodegradable natural materials such as burlap (e.g., wide weave burlap), jute, hemp, and/or sisal.

Surprisingly, structural components for sorbent products/devices made from biodegradable polypropylene have substantially equal properties to structural components made from non-biodegradable polypropylene. For example, like non-biodegradable polypropylene, biodegradable polypropylene is hydrophobic in nature, and thus is able to repel/shed water. Additionally, biodegradable polypropylene is substantially equal to non-biodegradable polypropylene in weight, density, strength, durability, chemical resistance, and floatation, even after adsorbing liquids such as hydrocarbons.

Moreover, structural components for use in sorbent products and devices made of biodegradable polypropylene, or natural materials such as jute, hemp, sisal and/or burlap (e.g., wide weave burlap), are durable in nature and thus will not rapidly decay upon deployment, but will decompose under standard landfill conditions within approximately two to three years, depending on the microbial environment within a particular landfill.

Biodegradable and Landfill Friendly Sorbents

The present invention further provides for the manufacture of sorbents for use in sorbent products and devices composed of one or more of the following biodegradable materials: (1) biodegradable polypropylene, (2) raw, short-fiber cotton, (3) perlite (e.g., expanded perlite), (4) a combination of raw, short-fiber cotton and perlite (e.g., expanded perlite), or (5) a combination of biodegradable polypropylene, raw short-fiber cotton, and perlite (e.g., expanded perlite).

Biodegradable Polypropylene Sorbents

The present invention provides for the use of biodegradable polypropylene in sorbent products and devices that are suitable for use in hydrocarbon, universal and/or hazmat applications, such as booms, bilge booms, socks, dikes, mats, rolls, pads, mops, rugs, pillows, pom-poms, skimmers, sweeps and wipes.

Biodegradable polypropylene can be used to manufacture melt-blown and spun-bond fibers in the same manner as non-biodegradable polypropylene. The difference is that products made with biodegradable polypropylene will decompose in approximately two to three-years under either aerobic or anaerobic landfill conditions, depending on the microbial environment within the particular landfill. The impact on the environment is therefore greatly minimized.

In general, oil-based synthetic polymers such as polypropylene are not readily biodegraded in the natural environment. Research indicates that oxidative enzymes such as dehydrogenase, peroxidase and monooxygenase can be responsible for the oxidation of long polypropylene polymer chains. Once these chains are broken, the resulting materials can enter microbial cells and be utilized as an energy source.

Regardless of the specific system used to promote biodegradation, decomposition of the polymer chains initiates the process and encourages microorganisms to use the resulting carbon sources as catabolites. The end result of this landfill biodegradation is the creation of water, carbon dioxide and other natural products.

Sorbents made from biodegradable polypropylene are surprisingly equal to sorbents composed of traditional, non-biodegradable polypropylene in 1) ability to repel/shed water (hydrophobic in nature), 2) hydrocarbon adsorbency (petrophilic in nature), 3) floatation properties, and 4) ability to endure chemical and environmental stresses.

Cotton

The present invention provides for the use of raw, short-fiber cotton as sorbents in sorbent products and devices that are suitable for use in hydrocarbon, universal and/or hazmat applications, such as booms, bilge booms, socks, dikes, mats, rolls, pads, mops, rugs, pillows, pom-poms, skimmers, sweeps and wipes.

Cotton is a biodegradable and sustainable product. Cotton selectively adsorbs oil due to the natural wax coating on its fibers, which is structurally similar to hydrocarbons. The small diameter fibers of cotton provide a large surface area and retain significant amounts of oil for later recovery. Cotton is also an excellent sorbent for non-aggressive liquids and aggressive liquids such as acids, bases, ketones, alcohols, and chlorinated solvents.

Re-ginning cotton gin residue or reginning the seeds removed as a result of initial ginning removes much of the stem, bole, seed and other non-fibrous material. What remains is raw, short-fiber cotton (lint).

A 2010 position statement published in the American Society of Agricultural and Biological Engineers referenced research performed by William Stanley Anthony of the U.S. Department of Agriculture which demonstrated that cotton lint adsorbs up to 80 times its weight in oil (Anthony, W. S. 1994. Absorption of Oil With Cotton Products and Kenaf. Applied Engineering in Agriculture 10(3): 357-361). Further, an article published in Cotton Today entitled “The Role of Cotton in the Deepwater Horizon Clean-Up” stated that “ . . . in real world trials using crude oil, raw cotton adsorbs roughly twice as much by weight as polypropylene (petroleum based) wipes and booms that are more generally used in the United States.” Based on these reports, lint-containing cotton fiber and fibrous gin waste products (i.e., raw, short-fiber cotton (lint)) appear to be superior to many of the synthetic materials currently being used in oil clean-up situations, including non-biodegradable polypropylene.

Raw, short-fiber cotton is more petrophilic than other natural organic materials that are currently employed in sorbent products. A small percentage of sorbent products on the market today use natural, organic products, such as corn cob, cellulose, kenaf and straw, and certain types of cotton, as sorbents. These materials have been proven to be impractical for use in booms and other sorbent products that are deployed in water. In such applications, these materials rapidly adsorb water instead of oil or other hydrocarbons, which compromises the ability of the products to float. Additionally, organic sorbents, especially corn cob, kenaf, straw and cellulose products, often leach naturally occurring sugar compounds into the aquatic environments when used in booms and other sorbent products deployed in aquatic environments. These sugars are capable of triggering numerous undesirable biological environmental problems such as algal blooms. In addition, once deployed in water environments, the corn cob, knead, straw and cellulose products sorbents tend to start decomposing immediately upon deployment.

Raw, short-fiber cotton is biodegradable and landfill friendly. The cotton plant material contains naturally occurring non-pathogenic bacteria and microflora that will substantially increase the rate of decomposition of both the hydrocarbons and the cotton itself, producing water, carbon dioxide and minerals as byproducts. Once the food sources are eliminated, the microorganisms will die off with no negative impact on the landfill.

Additionally, in contrast to other organic sorbents, sorbents that include raw, short-fiber cotton, as described herein, will not leach sugars and other harmful materials into waterways, and thus do contribute to environmental damage like other organic sorbents. Moreover, the raw, short-fiber cotton sorbents in accordance with the present invention can be incinerated without the production of dangerous or noxious fumes or other adverse environmental effects.

Perlite

The present invention provides for the use of expanded perlite as a sorbent in sorbent products and devices that are suitable for use in hydrocarbon, universal and/or hazmat applications, such as booms, bilge booms, socks, dikes, mats, rolls, pads, mops, rugs, pillows, pom-poms, sweeps, skimmers and wipes. Optionally, the perlite is coated with silicone. When coated with silicone, the petrophilic nature of perlite may be increased/expanded.

Perlite is a generic name for a naturally occurring siliceous volcanic rock. Perlite is inorganic, has a neutral pH, and is non-combustible. Perlite is classified as chemically inert and does not shrink, warp or slump. It is best known for its use as an aeration and product for gardens, as industrial insulation material and as a packing agent.

When heated, perlite expands four to twenty times its original volume, creating lightweight particles with countless internal cells and a high surface area. Thus, sorbent products employing expanded perlite, in accordance with the present invention, will assist in aeration of the landfill and promote degradation of organic debris. Thus, sorbent products that include perlite, as described herein, will contribute to the viability and health of landfills. Alternatively, sorbent products containing perlite can be reduced by incineration without the production of dangerous or noxious fumes or other adverse environmental effects.

The use of perlite sorbents provides several additional benefits when used in the manufacture and development of sorbents for use in hydrocarbon sorbent products/devices. For example, perlite contributes to the overall buoyancy of sorbent products deployed in aquatic environments, such as booms, even after the adsorption of substantial amounts of hydrocarbons. This is due to perlite's low weight-to-density ratio. Maintaining buoyancy is important for sorbent products deployed in aquatic environments because products that do not float adequately allow contaminated water to wash over them due to tidal and wind action. Moreover, the use of perlite provides flexibility to sorbent products such as booms and socks, which facilitates storage, shipping, deployment and recovery. Perlite is also a good sorbent for non-aggressive liquids and aggressive liquids such as acids, bases, ketones, alcohols, and chlorinated solvents.

Raw, Short-Fiber Cotton in Combination with Expanded Perlite

In a particular embodiment, the present invention provides for the use of raw, short-fiber cotton in combination with expanded perlite, in the manufacture and development of sorbents for use sorbent products and devices that are suitable for use in hydrocarbon, universal and/or hazmat applications, such as booms, bilge booms, socks, dikes, mats, rolls, pads, mops, rugs, pillows, pom-poms, sweeps, skimmers and wipes. Optionally, the perlite can be coated with silicone, which may increase the petrophilic nature of perlite and/or confer hydrophobic properties to the perlite, thereby reducing or eliminating the potential to adsorb water.

The combination of raw, short-fiber cotton and expanded perlite is an excellent sorbent for hydrocarbons, aggressive liquids and non-aggressive liquids. As such, it is suitable for use in hydrocarbon spills, as well as universal and hazmat applications.

It is recognized that the amount of hydrocarbon adsorbed by any product varies with the type of hydrocarbon, temperature, weather conditions and other factors. However, in controlled studies, raw, short fiber cotton adsorbed more oil than traditional, non-biodegradable polypropylene, and all other natural products tested.

In addition to the benefits of each of raw, short-fiber cotton and perlite, when used in sorbent products as described above, the combination of raw, short-fiber cotton and expanded perlite provides superior properties as compared to the individual components when used alone. For example, the combination of raw short-fiber cotton and expanded perlite employed in sorbents, in accordance with the present invention, allows for superior floatation properties in water as compared to the use of raw, short-fiber cotton, or perlite, alone. Additionally, the blend of raw short- fiber cotton and expanded perlite increases both oil adsorbency and floatation properties of the products while minimizing both dry and wet weights, as compared to the individual components when used alone.

Moreover, the cotton/ perlite sorbent identified in this invention can be incinerated to reduce the adsorbed oil, structural materials and cotton sorbent, without the production of dangerous or noxious fumes or other adverse environmental effects.

Biodegradable Polypropylene in Combination with Raw, Short-Fiber Cotton and/or Perlite

In another particular embodiment, the present invention provides for the use of biodegradable polypropylene, as described above, in combination with raw, short-fiber cotton and/or expanded perlite in the manufacture and development of sorbents for use in sorbent devices such as booms, bilge booms, socks, dikes, mats, rolls, pads, mops, rugs, pillows, pom-poms, sweeps, skimmers and wipes. The perlite may be optionally coated with silicone to increase the petrophilic nature of perlite and/or confer hydrophobic properties to the perlite, thereby reducing or eliminating the potential to adsorb water)

In addition to the benefits of each of biodegradable polypropylene, raw, short-fiber cotton, and perlite, when used in sorbent products, as described above, the combination of biodegradable polypropylene, raw, short-fiber cotton and/or expanded perlite provides superior properties as compared to the individual components alone. For example, the combination of biodegradable polypropylene, raw short-fiber cotton and/or expanded perlite employed in sorbents, in accordance with the present invention, allows for maximum floatation in water as compared to the use of biodegradable polypropylene, raw, short-fiber cotton, or perlite, alone. Additionally, the combination of biodegradable polypropylene, raw short- fiber cotton and/or expanded perlite maximizes both oil adsorbency and floatation of the products while minimizing both dry and wet weights as compared to the individual components when used alone.

Liquid Sorbent Products and Devices

The present invention provides sorbent products and devices, including but not limited to booms, bilge booms, socks, dikes, mats, rolls, pads, mops, rugs, pillows, pom-poms, sweeps, skimmers and wipes, that include one or more fully biodegradable structural components and/or sorbents, as described in detail above. Such sorbent products and devices are suitable for use in hydrocarbon, universal and/or hazmat applications. In a particular embodiment, the sorbent products/devices of the invention include both structural components and an internal sorbent, each of which are fully biodegradable.

The skilled artisan will readily appreciate that the sorbent products of the invention can be manufactured using biodegradable structural components and/or biodegradable sorbents in any configuration, size, and/or shape.

By way of example, a boom including biodegradable structural components and/or a biodegradable internal sorbent, in accordance with the present invention, can include one or more of the following features: it can be from several inches to 300 feet in length; from 2 inches to 10 feet in diameter; be in a series of lengths of boom with the segments of boom being strung together; include a sorbent comprised of a mixture of biodegradable polypropylene alone, or in combination with raw, short-fiber cotton and/or silicone coated perlite, in proportions ranging from 10% to 90% of each of those components; be encased in a biodegradable polypropylene or other suitable biodegradable containment sheet or casing (e.g., made from burlap, hemp, jute or sisal); include or not include a biodegradable rope of various types of construction (e.g., made from burlap, hemp, jute or sisal); be foldable or not foldable; include one or more hydrocarbon degrading bacteria; and/or be linkable or used as a single segment of boom (e.g., in the bilge of a boat or ship).

As a further example, a mat including biodegradable structural components and/or a biodegradable internal sorbent and/or simply composed of any form of biodegradable material, in accordance with the present invention, can include one or more of the following features: it can be from several square inches to several hundred square feet in area; of any thickness; include a sorbent comprised of a mixture of biodegradable polypropylene alone, or in combination with raw, short-fiber cotton and/or perlite, in proportions ranging from 10% to 90% of each of those components; include a sorbent entirely composed of biodegradable polypropylene; be encased in a biodegradable polypropylene or other suitable biodegradable containment casing (e.g., made from burlap, hemp, jute or sisal); be foldable or not foldable; include one or more hydrocarbon degrading bacteria; and/or be linkable or used as a single segment of mat.

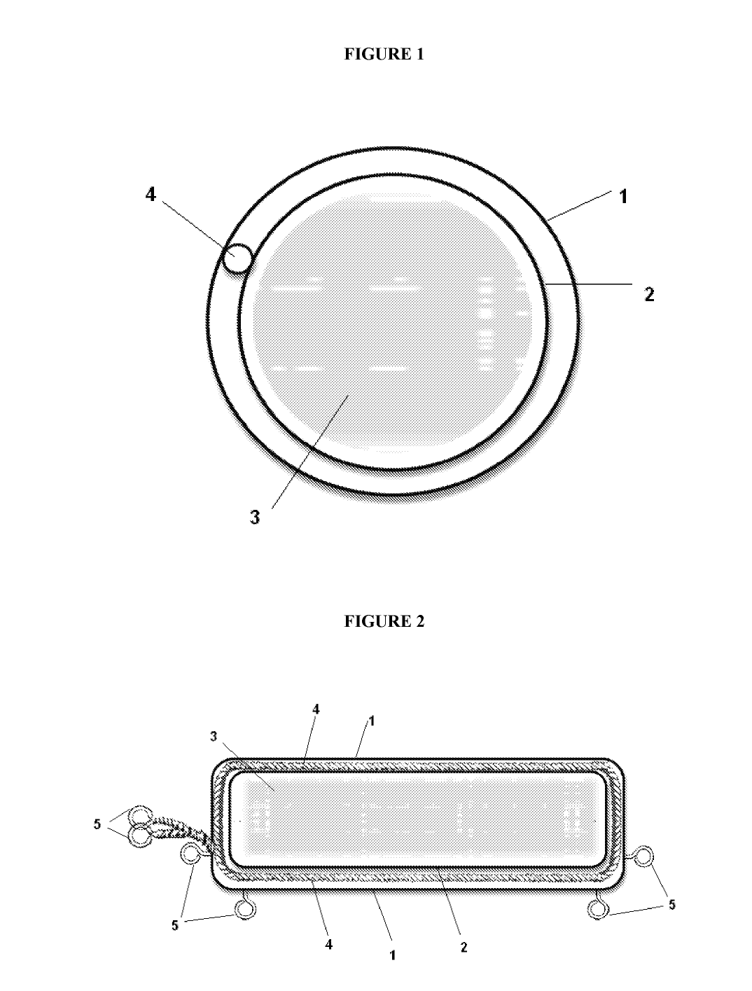

Exemplary embodiments of sorbent products in accordance with the invention are depicted in FIGS. 1-4. For example, FIG. 1 depicts a cross-section of a biodegradable boom in accordance with the invention. As shown in FIG. 1, the boom includes a protective scrim (cover)) 1 made of biodegradable polypropylene or natural fiber, a casing 2 made of biodegradable polypropylene or natural fiber, an internal sorbent 3 composed or raw, short-fiber cotton, perlite and/or biodegradable polypropylene in combination or as individual components, and a biodegradable rope 4 extending the length of the boom. FIG. 2 depicts a lengthwise cross section of the exemplary boom depicted in FIG. 1. As depicted in FIG. 2, a boom in accordance with the invention can further include metal connectors 5 for linking multiple booms together. The metal connectors are preferably removable such that they can be removed prior to disposal of the boom in a landfill.

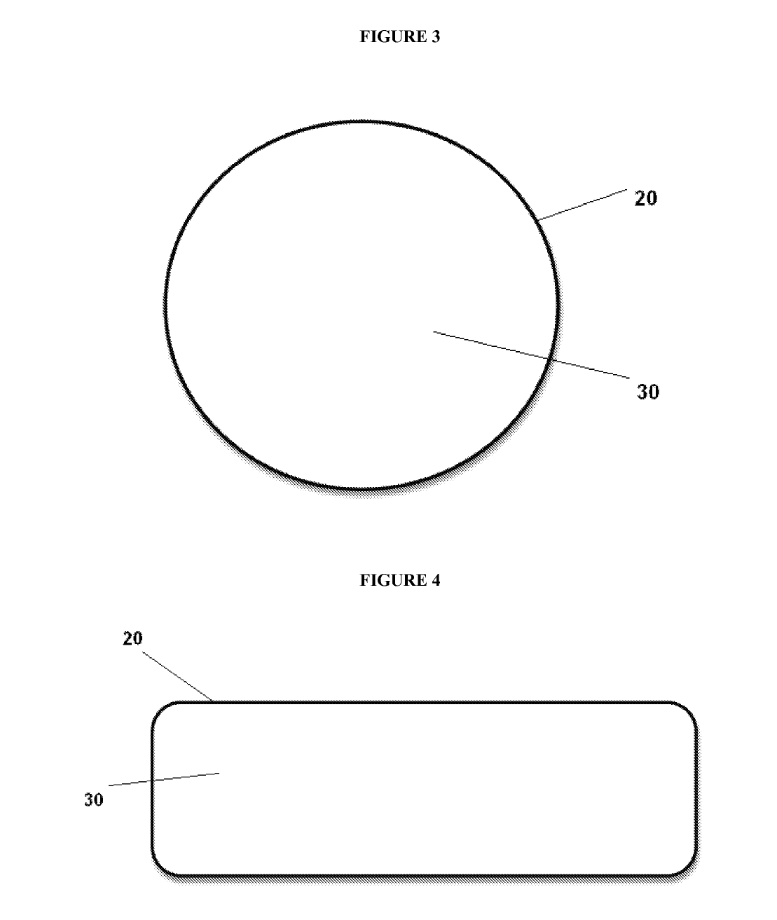

FIG. 3 depicts a cross-section of an exemplary embodiment of a sock in accordance with the invention. As shown in FIG. 3, the sock includes a casing 20 made of biodegradable polypropylene or natural fiber, and an internal sorbent 30 composed or raw, short-fiber cotton, perlite and/or biodegradable polypropylene in combination or as individual components. FIG. 4 depicts a lengthwise cross-section of the boom depicted in FIG. 3.

Deployment of the biodegradable sorbent products/devices according to the invention is accomplished in a manner that is similar or the same as the deployment of conventional existing sorbent products/devices. Deployment of booms can be to protect a shoreline and contain and adsorb an oil spill. The configuration of the shoreline deployment may include, but are not limited to Containment, Deflection, Cascade and Exclusion. The boom may also be towed by a vessel or vessels in order to capture oil on the open water.

Removal or extraction of biodegradable sorbent products/devices according to the invention is accomplished in a manner that is similar or the same as the removal of conventional sorbent products. Once the biodegradable sorbent products/devices of the invention are extracted, they can be directly disposed of in their entireties, after removal of any metal connectors, (i.e., without removing the ropes, casings, scrims, etc.) in a landfill as, where they will naturally degrade over time (e.g., 1-3 years). Alternatively the biodegradable products/devices according to the invention can be incinerated without producing hazardous or noxious fumes, or other adverse environmental effects.

It should be understood the foregoing detailed description is for purposes of illustration rather than limitation of the scope of protection accorded this invention, and therefore the description should be considered illustrative, not exhaustive. The scope of protection is to be measured as broadly as the invention permits. While the invention has been described in connection with preferred embodiments, it will be understood that there is no intention to limit the invention to those embodiments. On the contrary, it will be appreciated that those skilled in the art, upon attaining an understanding of the invention, may readily conceive of alterations to, modifications of, and equivalents to the preferred embodiments without departing from the principles of the invention, and it is intended to cover all these alternatives, modifications and equivalents. Accordingly, the scope of the present invention should be assessed as that of the appended claims and any equivalents falling within the true spirit and scope of the invention.

What is claimed is:

1. A device for containing and adsorbing a liquid substance in terrestrial or aquatic environments, comprising one or more structural components that are biodegradable.

2. The device of claim 1, wherein the liquid substance is a hydrocarbon, an aggressive liquid, or a non-aggressive liquid.

3. The device of claim 2, wherein the hydrocarbon is selected from the group consisting of crude oil, refined oil, kerosene, gasoline, machine oil, or any combination thereof.

4. The device of claim 2, wherein the aggressive liquid is selected from the group consisting of an acid, a base, an alcohol, a ketone, a solvent, a detergent, and any combination thereof.

5. The device of claim 2, wherein the non-aggressive liquid is selected from the group consisting of water, milk, juice, blood, and any combination thereof.

6. The device of claim 1, wherein the device is in a form selected from the group consisting of a boom, a bilge boom, a sock, a dike, a mat, a roll, a pad, a mop, a rug, a pillow, a pom-pom, a skimmer, a sweep a wipe, or any combination thereof.

7. The device of claim 1, wherein the one or more structural components include at least one of a casing, a rope, a scrim, or any combination thereof.

8. The device of claim 1, wherein the one or more structural components are comprised of a biodegradable material selected from the group consisting of biodegradable polypropylene, burlap, jute, hemp, sisal, or any combination thereof.

9. The device of claim 1, further comprising a biodegradable sorbent.

10. The device of claim 9, wherein the biodegradable sorbent is comprised of a material selected from the group consisting of:
a) biodegradable polypropylene;
b) a combination of raw, short-fiber cotton and perlite; and
c) a combination of biodegradable polypropylene, raw, short-fiber cotton, and perlite.

11. A biodegradable sorbent for use in a sorbent product or device, wherein the biodegradable sorbent is comprised of a material selected from the group consisting of:
a) biodegradable polypropylene;
b) a combination of raw, short-fiber cotton and perlite; and
c) a combination of biodegradable polypropylene, raw, short-fiber cotton, and perlite.

12. A device for containing and adsorbing a liquid substance in a terrestrial or aquatic environment and aggressive liquids in terrestrial environments comprising one or more structural components and a sorbent that are each biodegradable.

13. The device of claim 12, wherein the liquid substance is a hydrocarbon, an aggressive liquid, or a non-aggressive liquid.

14. The device of claim 13, wherein the hydrocarbon is selected from the group consisting of crude oil, refined oil, kerosene, gasoline, machine oil, or any combination thereof.

15. The device of claim 13, wherein the aggressive liquid is selected from the group consisting of an acid, a base, an alcohol, a ketone, a solvent, a detergent, and any combination thereof.

16. The device of claim 13, wherein the non-aggressive liquid is selected from the group consisting of water, milk, juice, blood, and any combination thereof.

17. The device of claim 12, wherein the device is in a form selected from the group consisting of a boom, a bilge boom, a sock, dike, a mat, a roll, a pad, a mop, a rug, a pillow, skimmer, sweep, pom-pom or any combination thereof.

18. The device of claim 12, wherein the one or more structural components include at least one of a casing, a rope, a scrim, or any combination thereof.

19. The device of claim 12, wherein the one or more structural components are comprised of a biodegradable material selected from the group consisting of biodegradable polypropylene, burlap, jute, hemp, sisal, or any combination thereof.

20. The device of claim 12, wherein the sorbent is comprised of a material selected from the group consisting of:
a) biodegradable polypropylene;
b) a combination of raw, short-fiber cotton and perlite; and
c) a combination of biodegradable polypropylene, raw, short-fiber cotton, and perlite.
